(19) United States
(12) Patent Application Publication
(10) Pub. No.: US 2008/0138876 A1
(43) Pub. Date: Jun. 12, 2008

(54) TRANSFECTION IN ELECTRONICALLY DRIVEN CONTINUOUS FLOW

(75) Inventor: Charles W. Ragsdale, Concord, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 11/609,196

(22) Filed: Dec. 11, 2006

(51) Int. Cl. C12N 13/00; C12M 1/00

(52) U.S. Cl. 435/173.1; 435/283.1

(57) ABSTRACT

Biological cells and other membranous structures are transfected in a flow-through system by using a moving charge pattern on a longitudinal wall of a channel to cause the cells to travel through the channel due to an electrostatic interaction between the cells and the moving charge pattern. As the cells travel through the channel, they pass a transmitter that emits transfection energy sufficient to make the cell membranes permeable such that exogenous species in the fluid in which the cells are suspended will enter the cell interiors.

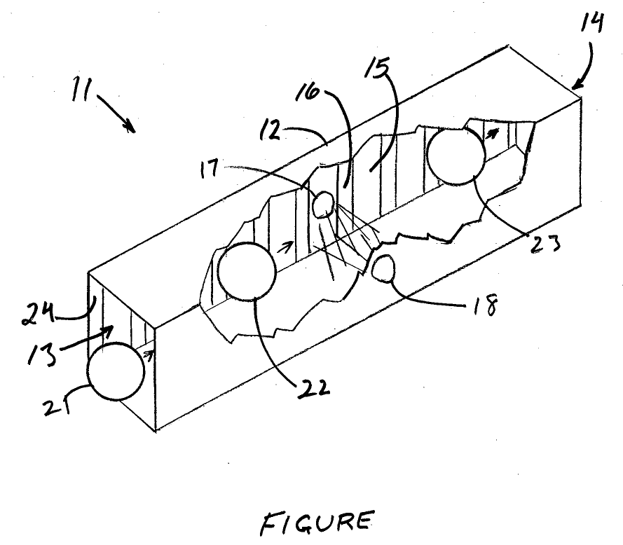

### BACKGROUND OF THE INVENTION

1. Field of the Invention

This invention lies in the field of transfection of membranous structures such as biological cells, liposomes, and vesicles with species that are exogenous to the structures. In particular, this invention relates to the mobilization of the membranous structures to produce a continuous-flow transfection system.

2. Description of the Prior Art

Transfection is of value to research biologists and biochemists in the performance of various investigations and procedures, including siRNA experiments and research using cDNA libraries, and various other clinical and research procedures. Some of the most advanced transfection technology is that involving electroporation, i.e., the application of electric field pulses through a suspension of the structures in a liquid solution of the exogenous species to render the membranes of the structures temporarily porous and thereby allow the species to penetrate the membrane. As the value of transfection is increasingly recognized and it use expands, certain concerns have limited its applicability. One such concern is the efficiency of the procedure, which is generally low and highly variable due to the tendency of the membranous structures to aggregate, the different orientations of the structures and the differences in exposure of the structures to the electric field with different orientations, and the shielding effect of individual structures which limits the exposure of the shielded structures to the field and to the exogenous species.

Another concern is throughput, particularly in situations where the transfection is to be performed on large volumes of cells or other membranous structures. Certain high-throughput applications are those involving multitudes of samples where different cell types or different exogenous species, or both, are to be subjected to the procedure simultaneously. To accomplish this, electroporation plates that accommodate large numbers of samples have been designed. Descriptions of such plates are found in International Patent Application Publication No. WO 2004/050866 A1, entitled “Large-Scale Electroporation Plates, Systems, and Methods of Use” (Genetronics, Inc., applicant; Gamelin, A., et al., inventors), published under the Patent Cooperation Treaty on Jun. 17, 2004; and in U.S. Provisional Patent Application No. 60/771,994, filed Feb. 10, 2006, entitled “Apparatus for High-Throughput Electroporation” (inventors Ragsdale, C. W., et al.) and commonly owned herewith. Other high-throughput applications are those that simply involve a large volume of membranous structures, more for example than can be accommodated in a single electroporation cuvette.

In the majority of the literature on electroporation and transfection in general, and all of the commercially available electroporation systems, the procedure is performed in cuvettes in a batchwise format. With the high degree of manipulation and repetition involved in batchwise procedures, together with the size limitations of the typical cuvette, the processing of large volumes of sample and large numbers of structures is costly and prone to error. Continuous use of an electroporation chamber designed for batchwise use entails a risk of overheating of the chamber and irreparable rupture of the membranes. Continuous-flow systems have been contemplated but with only limited application. Descriptions of continuous flow systems appear in Nicolau et al. (CBR Laboratories, Inc.), U.S. Pat. No. 5,612,207, “Method and Apparatus for Encapsulation of Biologically-Active Substances in Cells,” issue date Mar. 18, 1997, and Meserol, P. (EntreMed, Inc.), U.S. Pat. No. 6,090,617, “Flow Electroporation Chamber With Electrodes Having a Crystalline Metal Nitride Coating,” issue date Jul. 18, 2000. The electrodes in these patents are elongated strip electrodes, and electroporation is achieved by pumping the cells through the space between the electrodes, using a simple mechanical pump. The electroporation rate is limited by the pump rate, and there is little or nor control over such factors as the density of the suspension at any particular point in the flow path and differences in the exposure of individual cells to the electric field. A description of another moving system is found in Acker, J. L., et al., United States Patent Application Publication No. US 2004/0029240 A1, publication date Feb. 12, 2004. The system used b Acker et al. involves moving electrodes and is not a flow-through system. The purpose of the moving electrodes is to impose a shear stress on the cells to cause them to continuously change their orientation.

Of further potential relevance to the background of the present invention is the use of electromagnetic radiation, such as pulses of light, to achieve transfection. In a manner analogous to electroporation, exposure of a membranous structure to a pulse of light energy can result in a transient permeabilization of the membrane without rupoture of the membrane. As in electroporation, this is done while the cell is suspended in a solution of a molecule that is exogenous to the cell, thereby allowing the molecule to enter the cell through the permeated membrane. A description of this technique is found in Koller, M. R., et al. (Oncosis LLC), U.S. Pat. No. 6,753,161 B2, “Optoinjection Methods,” issue date Jun. 22, 2006. The transient permeabilization effect is performed while the cells are “substantially stationary.”

### SUMMARY OF THE INVENTION

The present invention resides in a system and method for the transfection of electrically charged membranous structures in a continuous-flow format by utilizing the electrical charge on the structures to convey the structures through a channel and past a transmitter of transfection energy in the channel. By virtue of their electrical charge, the structures are attracted to opposing electrical charges on a longitudinal wall within the channel, the opposing electrical charges being imposed on chargeable surface regions that are arranged in a linear array and charged in succession to create a moving charge pattern along the wall. The moving charge pattern allows the travel of the membranous structures to be controlled to such an extent that the membranous structures can be made to establish moving contact with, or very close proximity to, the wall and to travel in a single file past the transfection energy transmitter where they will undergo transfection either one at a time or in groups of preselected size at preselected time and spatial intervals. As in conventional transfection, the structures are suspended in a solution of the exogenous species, and the moving charge pattern also allows each structure to be exposed to the same electric field without aggregation of the structures or shielding of one structure by another. Transfection that is substantially uniform among all of the structures at a high rate of efficiency is thus achieved, with sufficient control over the transfection conditions that destruction of the structures due to excessive energy from the transmitter is minimal, if not eliminated entirely. Automated electronic control over the charging of the chargeable surface regions on the wall also allows the system to accommodate membranous structures of different sizes and dimensions by selecting the number and spacing of the regions to be charged in the moving pattern, to vary the spacing between adjacent membranous structures, and to vary the number of structures that are exposed to the transmitter at any point in time, i.e., whether transfection be performed on only one structure at a time or more than one.

These and other operations, functions, and advantages of this invention are explained in further detail below.

### BRIEF DESCRIPTION OF THE DRAWING

The FIGURE is a perspective view of a transfection channel in accordance with the present invention with a portion of the channel wall removed to show the interior.

### DETAILED DESCRIPTION OF THE INVENTION AND PREFERRED EMBODIMENTS

The membranous structures to which the present invention is applicable are bodies that are at least of macromolecular dimensions and include an enclosing membrane that is impenetrable to the species of interest under normal conditions. Examples of such membranous structures are liposomes, vesicles, organelles, and biological cells. Biological cells include both prokaryotic and eukaryotic cells, and can be animal cells, plant cells, yeast cells, human cells, bacteria, or any other similar structures. The electrostatic charges on the membranous structures can be either naturally occurring or added by surface functionalization or complexation. Many biological cells, for example, bear a negative surface charge in their natural form.

The term “exogenous species” is used herein to denote any molecule or cluster of molecules that is not native to or otherwise present in the membranous structures, or is present inside the structure but in a limited quantity or at a limited concentration and whose quantity or concentration within the structure is to be increased by transfection in accordance with this invention. Examples of classes of exogenous species are nucleic acids, polypeptides, carbohydrates, lipids, and small molecules in general. Examples of nucleic acids are RNA, expression plasmids, expression cassettes and other expressible DNA. Examples of polypeptides are antibodies, antibody fragments, enzymes, and proteins in general. Examples of carbohydrates are non-naturally occurring metabolites such as isotopically labeled sugars, and polysaccharides such as labeled dextrans. Liposomes may serve as exogenous species when the membranous structures are bodies larger than liposomes. Examples of small molecules are drugs, dyes, and ligands for endogenous receptors.

The term “transfection energy” is used herein to denote any form of energy applied to a membranous structure that will render the membrane reversibly porous or otherwise permeable for a limited period of time sufficient to allow exogenous species in the suspending liquid to penetrate the membrane and enter the interior of the structure, and to do so without irreparably rupturing the membrane or otherwise causing permanent damage to the structure. Examples of transfection energy are electrical energy (resulting in electroporation), light energy (both from a laser and from non-laser sources), thermal energy, RF energy, ultrasound, and electron beam energy. Preferred forms of transfection energy are electrical energy and laser light energy, applied either individually or in combination. Electrical energy (electroporation) is particularly preferred. The “transfection energy transmitter” is any device or component that will create a field of transfection energy, preferably one that is focused within a spatial volume of dimensions that are limited to achieve transfection in a preselected number of membranous structures. The field can be small enough to accommodate only one structure at a time, or broad enough to accommodate a limited plurality such as two or more structures, or it can be a ray of energy sufficiently narrow to strike only one structure. Transmitters that are known in the art for each particular type of energy can be used. For electroporation, the transmitters can be electrodes; for light or thermal energy, the transmitters can be laser diodes. Other transmitters for these and other forms of transfection energy will be apparent to those skilled in the art.

The electrically chargeable surface regions on the longitudinal wall of the channel are fixed, stationary regions that can be individually and selectively charged, i.e., rapidly switched between charged and electrically neutral, or between positively charged, negatively charged, and neutral, by switching means external to the wall or to the channel. Each region occupies a fixed position on the wall and can be formed by attachment of an electrode to the surface of the wall, incorporation of an electrode material in the construction of the wall, or doping the wall with ionic species as in conventional semiconductor fabrication technology. In certain embodiments, the wall is formed of silicon or other semiconductor material and the regions are strip areas of the wall doped with chargeable ions.

The moving charge pattern on the wall preferably consists of two or more regions bearing charges that attract the membranous structure, and more preferably two or more such regions in addition to one or more regions bearing charges that repel rather than attract the membranous structure. The region(s) bearing the repelling charge will be positioned upstream (i.e., at the trailing end) of the region(s) bearing the attracting charge to help propel the membranous structure forward in the direction of travel through the channel, the two charges thereby imposing both a pushing force and a pulling force in combination on each passing structure. The use of two regions of attracting charge in the charge pattern serves to provide optimal control of the positions of the membranous structures at all points during their travel through the channel, stabilizing the structure across the charged regions when the spacing between the charged regions is approximately equal to or slightly smaller than the length or diameter of the membranous structure. In certain embodiments, the system is adaptable by allowing the operator to select among different charge patterns to accommodate membranous structures of different sizes. Two regions bearing attractive charges can thus be separated by one or more uncharged regions in the charge pattern, the number of intervening uncharged regions determining the spacing of the charged regions. In most cases, best results will be obtained with a center-to-center spacing of from about 0.1 micron to about 10 microns, and preferably from about 0.3 micron to about 3 microns, between regions bearing the attractive charge.

The same surface regions that move the membranous structures can also be used to transfect. This is explained in detail below. The surface regions can also serve as detectors of the sizes of the membranous structures. As a structure moves across adjacent surface regions, current can be passed through the regions, and the resistance to the current measured. The resistance when a cell or other membranous structure is touching a given surface region will differ from the resistance when no structure is touching the region. The number of adjacent regions that the structure is in contact with at a given point in time thus indicates the size of the structure. Size can also be detected by optical sensors, such as for example LEDs (light-emitting diodes) in conjunction with phototransistors positioned to receive the beams of light from the LEDs through the moving path of the membranous structures. Regardless of the mechanism, once the sizes of individual membranous structures are determined, the charge pattern can be adjusted to accommodate the structure size and thereby provide optimal control over the movement of the structures.

As noted above, the chargeable surface regions can be formed by integrated circuit techniques such as doping of metallization etched into a semiconductor surface. The electronic drivers that govern the charge pattern and its movement can be such commonly known components as transistors, IGBTs (insulated gate bipolar transistors) and power FETs (field effect transistors). As an example of a charging protocol to create a moving charge pattern, a first series of surface regions, for example, the four such regions at the entry to the channel, can be made positive to attract a biological cell, which bears a natural negative charge. When a cell is sensed by electrical or optical means as described in the preceding paragraph, or after a specified period of time has passed, the first region is turned off (switch to electrical neutrality) and then given a negative charge as the fifth region (previously neutral) is given a positive charge. This continues in succession down the array of surface regions.

The term “linear array” is used herein to indicate electrically chargeable regions that are arranged in a line, which can be either curved or straight, such that when the charge pattern is moved along them they direct the membranous structures along a unidirectional path of travel. In most cases, a straight-line array will be most convenient. Two or more parallel linear arrays can be present, doubling or otherwise multiplying the capacity of the channel and the rate of transfection.

The system can be designed to accommodate either a single structure at a time passing through the channel or multiple structures. When the channel is long enough to accommodate two or more structures, the charge protocol will include a number of moving charge patterns equal to the number of structures. The spacing between adjacent charge patterns will preferably be sufficient to avoid interference between successive structures in their movement through the channel and in their exposure to the transfection energy from the transmitter. A spacing equal to ten or more structure diameters, and preferably fifty or more, will provide the best results in most cases.

The charge pattern can be designed to cause the membranous structures to travel in a single file, double file, or more. Travel in a single file is generally sufficient in most applications and can be achieved by limiting the dimensions of the charged regions, the dimensions of the channel, or both. The travel velocity and number of structures passing through the channel per unit time can also vary. Preferably, the rate of travel is high enough to cause ten or more structures per second to pass the transfection energy transmitter, preferably 100 to 10,000 structures per second, and most preferably 300 to 3,000 structures per second.

The dimensions of the channel will nevertheless be large enough to allow the structures to flow freely through the channel without clogging the channel. A channel width or diameter of at least about 10 microns, preferably about 20 microns or greater, will be suitable in most cases, particularly for biological cells.

The transfection energy transmitter is positioned at a fixed location in the channel so that membranous structures during the course of their travel through the channel will come within the range of the transmitter. When the transmitter is a pair of electrodes to cause transfection by electroporation, the electrodes can be a dedicated pair of electrodes, either on the same side of the channel or on opposing sides. For electrodes on the same side of the channel, two of the chargeable surface regions, either adjacent or in close proximity, can also serve as the electroporation electrodes by imposing a higher voltage between them for electroporation. For example, when simply causing the travel of a structure, the two surface regions can be charged at the same polarity with a charge in the millivolt range, and when the structure is in position for electroporation, the two regions can be charged at opposite polarities with charges in the volt range. In view of the very small dimensions of the system and the close proximity of the membranous structures to the electrodes, a typical voltage range for electroporation will be within the range of 0.3-30 V, preferably 1-5 volts, and this will typically be 10 to 1,000 times the voltage for travel. When electroporation electrodes are used that are distinct from the moving charge pattern surface regions, the former can be placed between an adjacent pair of the latter. When laser diodes or other transmitters that produce temperature- or light-induced poration are used, they can likewise be placed on one side of the channel or on opposing sides, and most effectively between an adjacent pair of chargeable surface regions. Laser diodes will require little or no optics in view of their close proximity to the membranous structures.

While the features defining this invention are capable of implementation in a variety of constructions, the invention as a whole will be best understood by a detailed examination of a specific embodiment. One such embodiment is shown in the attached FIGURE.

The FIGURE depicts a continuous-flow transfection apparatus 11 which includes a channel 12 shown with parts of the wall removed to make the interior of the channel visible. The channel is open at both ends, with one end designated an entry end 13 and the other an exit end 14. The inner surface 15 of one longitudinal wall contains a series of regularly spaced regions 16 that serve as the chargeable regions, with adjacent regions spaced apart from each other. Transfection energy transmitters 17, 18 are positioned on opposing sides of the channel, one 17 on the same wall as the chargeable regions and between two adjacent chargeable regions, and the other 18 directly opposite on the opposing wall. Three membranous structures (negatively charged biological cells) 21, 22, 23 are shown moving through the channel in the direction indicated by the arrows. The moving charge pattern in this case consists of three adjacent surface regions, the first located closest to the entry end 13 and bearing a negative charge that repells the cells, and the second and third bearing a positive charge that attracts the cells. To draw a cell into the channel, the region 24 nearest the entry end is positively charged. As the second and third regions become positively charged, the region 24 nearest the entry end is given a negative charge to urge the cell further into the channel. The charge pattern then travels through the channel, drawing the cell with it, past the transmitters 17, 18.

While the foregoing description describes various alternatives to the components shown in the FIGURES, still further alternatives will be apparent to those who are skilled in the art and are within the scope of the invention.

In the claims below, the terms “a” and “an” are intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A method for transfecting a plurality of electrostatically charged membranous structures with species exogenous to said structures, said method comprising:
(a) introducing a dispersion of said membranous structures in a liquid solution of said exogenous species into a channel to which is mounted a transfection energy transmitter, said channel comprising a longitudinal wall with a linear array of electrically chargeable surface regions;
(b) electrically charging said surface regions in succession to produce electrostatic forces between said surface regions so charged and said membranous structures and to thereby cause said membranous structures to travel in a direction along said longitudinal wall and past said transfection energy transmitter; and
(c) as each said membranous structure passes said transfection energy transmitter, actuating said transfection energy transmitter to achieve said transfection.

2. The method of claim 1 wherein step (b) comprises imposing a moving electrostatic charge pattern on said surface regions, said charge pattern comprising a membranous structure attracting charge on a first number of surface regions and a membranous structure repelling charge on a second number of surface regions upstream of said first number relative to said direction of travel.

3. The method of claim 2 wherein said first number of surface regions is two or more and said surface regions of said first number are adjacent.

4. The method of claim 2 wherein said first number of surface regions is two or more and said charge pattern comprises said membranous structure attracting charge on two surface regions separated by an uncharged region.

5. The method of claim 1 wherein said surface regions are sufficiently small to cause said membranous structures to travel past said transfection energy transmitter in a single file.

6. The method of claim 1 wherein said membranous structures are negatively charged biological cells and step (b) comprises imposing a positive charge to said surface regions in succession.

7. The method of claim 2 wherein said membranous structures are negatively charged biological cells and said membranous structure attracting charge is a positive charge and said membranous structure repelling charge is a negative charge.

8. The method of claim 1 wherein said transfection energy transmitter is a pair of electroporation electrodes.

9. The method of claim 8 wherein said electroporation electrodes are a selected pair of said electrically chargeable surface regions, and step (c) comprises imposing an electroporation potential between said selected pair.

10. The method of claim 9 wherein said electroporation potential is achieved by imposing charges on said selected pair of electrodes that are at least 10 times the charge imposed in step (b).

11. The method of claim 8 wherein said electroporation electrodes are positioned on opposing sides of said channel.

12. The method of claim 1 wherein said transfection energy transmitter is a laser diode.

13. The method of claim 1 wherein said transfection energy transmitter is a combination of a pair of electroporation electrodes and a laser diode.

14. The method of claim 1 wherein step (b) comprises electrically charging said surface regions in succession at a rate causing said membranous structures to travel singly past said transfection energy transmitter at a rate exceeding 10 structures per second.

15. The method of claim 1 further comprising detecting locations and sizes of said structures by measuring resistance to electric current at said surface regions.

16. The method of claim 1 further comprising detecting locations and sizes of said structures by interception of light beams through said channel.

17. The method of claim 1 wherein step (b) comprises electrically charging said surface regions in succession at a rate causing said membranous structures to travel singly past said transfection energy transmitter at a rate of from 100 structures per second to 10,000 structures per second.

18. Apparatus for subjecting a plurality of electrostatically charged bodies in succession to transfection, said apparatus comprising:
a channel to which is mounted a transfection energy transmitter, said channel bounded by a longitudinal wall bearing a linear array of electrically chargeable surface regions;
transfection means for energizing said transfection energy transmitter to create an energy field sufficient to cause transfection of said electrostatically charged bodies when said bodies are within said energy field; and
conveying means for conveying said electrostatically charged bodies in succession through said energy field by electrically charging said surface regions in succession to produce a moving charge pattern of electrostatic forces that attract said electrostatically charged bodies.

19. The apparatus of claim 18 wherein said transfection energy transmitter is comprised of electroporation electrodes and said energy field is an electric field.

20. The apparatus of claim 19 wherein said electroporation electrodes are a selected pair of said electrically chargeable surface regions and said transfection means are means for electrically charging said selected pair of surface regions to charges that are at least 10 times the charges applied by said conveying means.

21. The apparatus of claim 19 wherein said transfection energy transmitter is a laser diode and said energy field is a light energy field.

22. The apparatus of claim 19 wherein said transfection energy transmitter is a combination of electroporation electrodes and a laser diode and said energy field is a combination of an electric field and a light energy field.

23. The apparatus of claim 18 wherein said electrically chargeable surface regions are sufficiently small to cause said bodies to travel through said energy field in a single file.

24. The apparatus of claim 18 wherein longitudinal wall is a surface of a semiconductor material and said electrically chargeable surface regions are discrete doped domains in said semiconductor material.

25. The apparatus of claim 24 wherein said doped domains have center-to-center spacings of from about 0.1 micron to about 10 microns.

26. The apparatus of claim 24 wherein said doped domains have center-to-center spacings of from about 0.3 micron to about 3 microns.
